(19) United States
(12) Patent Application Publication
(10) Pub. No.: US 2025/0066380 A1
(43) Pub. Date: Feb. 27, 2025

(54) NOVEL FURANO-NAPHTHOQUINONE AND NOVEL PYRAZINONE COMPOUNDS FROM AMYCOLATOPSIS AND ACTIVITIES THEREOF

(71) Applicant: COUNCIL OF SCIENTIFIC AND INDUSTRIAL RESEARCH (AN INDIAN REGISTERED BODY INCORPORATED, New Delhi (IN)

(72) Inventors: Syed Gulam DASTAGER, Pune (IN); Abujunaid KHAN, Pune (IN); Madhukar Shyam SAID, Pune (IN)

(21) Appl. No.: 18/725,039

(22) PCT Filed: Feb. 9, 2023
(86) PCT No.: PCT/IN2023/050133

§ 371 Date: Jun. 27, 2024

(30) Foreign Application Priority Data
Feb. 18, 2022 (IN) 202211009245

(51) Int. Cl. C07D 493/14; A61K 31/343; A61K 31/513; A61P 31/04; A61P 33/06; A61P 35/00; C07D 239/36; C07D 493/04; C12N 1/20; C12P 17/12; C12P 17/16; C12R 1/01

(52) U.S. Cl. CPC C07D 493/14; A61K 31/343; A61K 31/513; A61P 31/04; A61P 33/06; A61P 35/00; C07D 239/36; C07D 493/04; C12N 1/205; C12P 17/12; C12P 17/162; C12R 2001/01

(57) ABSTRACT

The present disclosure provides an isolated strain of Amycolatopsis sp. WGS_07 having accession number MCC 0218. The present disclosure provides a process for synthesizing furano-naphthoquinone and pyrazinone derivatives using a novel strain Amycolatopsis sp. . The novel compounds synthesized possesses activity against gram positive bacteria, malarial parasites, and various cancers.

### FIELD OF THE INVENTION

The present invention relates to a process of synthesizing novel furano-naphthoquinone derivatives and a novel pyrazinone using a strain Amycolatopsis sp. WGS_07. The present invention further relates to furano-naphthoquinone and pyrazinone derivatives showing activity against gram-positive bacterial infections, malarial infections, and cancer.

### BACKGROUND AND PRIOR ART OF THE INVENTION

Throughout the ages, natural products have been the most consistently successful source of lead compounds that have found many applications in the fields of medicine, pharmacy, and agriculture. Natural products have been the source of most of the antibiotics in current use for the treatment of various infectious diseases.

The increase in antibiotic resistance is a big challenge to human health management. This crisis has been epitomized by the spread of multidrug-resistant “ESKAPE” organisms (Enterococcus spp., Staphylococcus aureus, Klebsiella spp., Acinetobacter baumannii, Pseudomonas aeruginosa, and Enterobacter spp.). Natural products from microbial sources have played a pivotal role in developing antimicrobial drugs over several decades. The bacterial genus Amycolatopsis, a rare actinobacteria, belongs to the Pseudonocardiaceae family and is producers of the important clinical antibiotics rifamycin and vancomycin. The saalfelduracins, thioamycolmides, amycolasporins, dibenzoyls, thioalbamide A, and pradimicin have been isolated from different species of Amycolatopsis. Multiple growth conditions have been employed to maximize the secondary metabolite diversity by varying the parameters such as nutrients, time, temperature, pH, and aeration.

Quinones represent a class of metabolites that are naturally distributed in animals, plants and microorganisms. Quinones are involved in important phases of living beings, mainly at the levels of photosynthesis (plastoquinones), respiratory chains (ubiquinones) and for controlling action on blood coagulation (naphthoquinones-types of Vitamin K). Interest in quinones has intensified not only for the crucial role they play in the energy production by providing essential links in the respiratory chain of the cells but also because of its prominence in various pharmacological studies The bioactivities reported from quinones are anticancer, antibacterial, antifungal, antiviral, anti-inflammatory and antiparasitic Quinones are the source of many cytotoxic compounds of anthracycline group used in therapy of cancer like doxorubicin, daunorubicin and mitoxantrone. The cytotoxicity of these compounds are associated with the production of reactive oxygen species (ROS) and by the inhibition of the human DNA topoisomerase I and II.

Quinones are chemically classified based on the different arrangement of aromatic system that supports the quinone ring i.e., benzoquinones (benzene ring), naphthoquinones (naphthalene ring) and anthraquinones (anthracene ring, linear or angular). Naphthoquinones are further classified into two groups based on the arrangements for their carbonyl groups, 1,2-naphthoquinones with adjacent functional groups or 1,4-naphthoquinones with a space of two carbons between carbonyls. These isomers have difference in their physicochemical properties because of which there are difference in their pharmacological actions.

Several studies have been conducted for the production of naphthoquinone or its derivatives using various microorganisms; however, most of them are challenged with their high production cost and low yield. Regardless of many interesting characteristics and advantages shown by naphthoquinone, the requirements of producing it on a large scale increased dramatically.

### OBJECTS OF THE INVENTION

The main objective of the present invention is to provide a process for the production of Furano-naphthoquinone and pyrazinone derivatives using a novel strain Amycolatopsis sp. WGS_07. Accordingly, the naphthoquinone derivatives described herein exhibit potent activity against gram-positive bacterial infections, malarial infections, and cancer.

It is yet another object of the present invention to provide a pharmaceutical composition comprising Furano-naphthoquinone and pyrazinone derivatives used for the treatment of gram-positive bacterial infections, malarial infections, or cancer.

### SUMMARY OF THE INVENTION

In first aspect the present invention provides an isolated strain of Amycolatopsis sp. WGS_07 having accession number MCC 0218.

In another aspect of the present invention, the Amycolatopsis sp. WGS_07 shows 99.24% similarity to that of strain Amycolatopsis silviterrae and 99.1% to strain Amycolatopsis Vancoresmycina.

In another aspect of the present invention, the Amycolatopsis sp. WGS_07 is isolated from soil using serial dilution method in which 100 μl of dilutions is spread on ISP-2 (1% malt extract, 0.4% yeast extract, 0.4% dextrose, pH 7.0) agar plates and incubated for 10 days at 28° C.

In another aspect of the present invention, the naphthoquinone based compounds and pyrazinone based compounds synthesized from the Amycolatopsis sp. WGS_07 is having activity against gram-positive bacterial infections, malarial infections, and cancer.

In another aspect, the present invention relates to a process for the synthesis of naphthoquinone based compounds and pyrazinone based compounds from the isolated strain of Amycolatopsis sp. WGS_07, wherein the process comprises the steps of:
(a) isolating the strain Amycolatopsis sp. WGS_07 on MGYP agar plates for a period of 5-7 days at a temperature in a range of 25-30° C. to obtain the Amycolatopsis sp. WGS_07;
(b) fermenting the Amycolatopsis sp. WGS_07 using fermentation media in static condition for a period of 5-7 days at a temperature in a range of 25-30° C. to obtain broth mixture at a pH in a range of 6.8 to 7.2;
(c) extracting the broth mixture using suitable solvent to obtain organic phase and concentrating the organic phase to obtain a crude extract comprising naphthoquinone derivatives;
(d) purifying the crude extract using column chromatography to obtain fractions comprising naphthoquinone derivative; and
(e) subjecting the fractions into the preparative HPLC to obtain the pure naphthoquinone based compounds 1-3 and pyrazinone based compound 4, represented as:

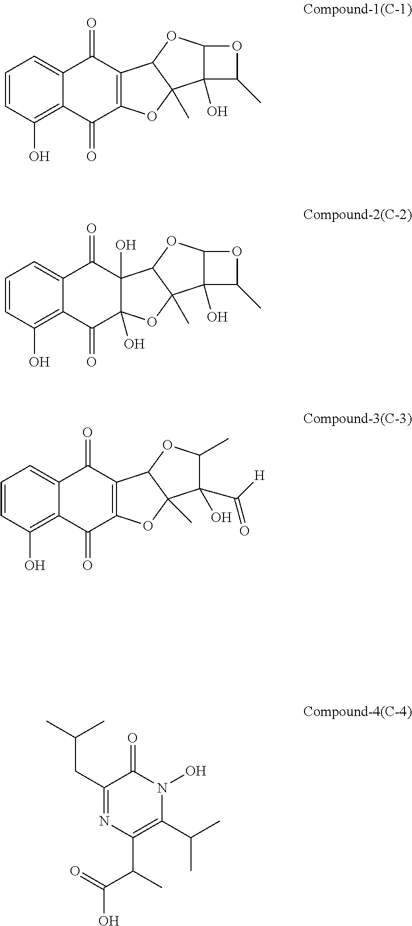

In yet another aspect of the present invention, the naphthoquinone compounds exhibit preferred activity and selectivity towards gram positive bacteria, with no activity against gram-negative bacteria at highest test concentration i.e., 128 μg/ml.

In an aspect of the present invention, the pyrazinone derivative C-4 exhibits antimalarial activity.

In another aspect of the present invention, a pharmaceutical composition comprising Furano-naphthoquinone and pyrazinone derivatives C-1, C-2, C-3 and C-4 is used for the treatment of gram-positive bacterial infections, malarial infections or cancer.

In another aspect, the present invention relates to a pharmaceutical composition comprising the naphthoquinone compounds.

These and other features, aspects, and advantages of the present subject matter will be better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

### BRIEF DESCRIPTION OF THE DRAWINGS

The invention has other advantages and features which will be more readily apparent from the following detailed description of the invention, when taken in conjunction with the accompanying drawings, in which:

FIG. 1 depicts the Amycolatopsis sp. WGS_07 on ISP-2 medium agar plate, in accordance with an embodiment of the present disclosure.

FIG. 2 depicts the phylogeny tree of Amycolatopsis sp. WGS_07 formed using the Neighbour Joining method, in accordance with an embodiment of the present disclosure.

FIG. 3 depicts the bioactivity screening of Amycolatopsis sp. WGS_07 strains for naphthoquinone derivatives using three fermentation mediums in plate, static, and shaking conditions, in accordance with an embodiment of the present disclosure.

FIG. 4 depicts the bioautography of crude extract done against S.aureus using shaking, static, and plate-based incubation conditions, in accordance with an embodiment of the present disclosure.

FIG. 5 depicts the day-wise activity of Amycolatopsis sp.WGS_07 supernatant against S. aureus, in accordance with an embodiment of the present disclosure.

FIG. 6 depicts the ORTEP view of compound C-1 showing the atom-numbering scheme, in accordance with an embodiment of the present disclosure.

FIG. 7 depicts the ORTEP view of compound C-4 showing the atom-numbering scheme, in accordance with an embodiment of the present disclosure.

FIG. 8 depicts the anticancer activity of C-1 against MDA-MB-231 (Breast cancer), A549 (Lung cancer) and HeLa (Cervical cancer) cell line, in accordance with an embodiment of the present disclosure.

FIG. 9 depicts the anticancer activity of C-2 against MDA-MB-231 (Breast cancer), A549 (Lung cancer) and HeLa (Cervical cancer) cell line, in accordance with an embodiment of the present disclosure.

FIG. 10 depicts the anticancer activity of C-3 against MDA-MB-231 (Breast cancer), A549 (Lung cancer) and HeLa (Cervical cancer) cell line, in accordance with an embodiment of the present disclosure.

FIG. 11 depicts the anticancer activity of C-4 against MDA-MB-231 (Breast cancer), A549 (Lung cancer) and HeLa (Cervical cancer) cell line, in accordance with an embodiment of the present disclosure.

### DETAILED DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

### Definitions

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The invention has other advantages and features which will be more readily apparent from the following detailed description of the invention, when taken in conjunction with the accompanying drawings.

While the invention has been disclosed with reference to certain embodiments, it will be understood by those skilled in the art that various changes may be made, and equivalents may be substituted without departing from the scope of the invention. In addition, many modifications may be made to adapt to a particular situation or material to the teachings of the invention without departing from its scope.

Throughout the specification and claims, the following terms take the meanings explicitly associated herein unless the context clearly dictates otherwise. The meaning of “a”, “an”, and “the” include plural references. The meaning of “in” includes “in” and “on.” Referring to the drawings, like numbers indicate parts throughout the view. Additionally, a reference to the singular includes a reference to the plural unless otherwise stated or inconsistent with the disclosure herein.

The tables, figures and protocols have been represented where appropriate by conventional representations in the drawings, showing only those specific details that are pertinent to understanding the embodiments of the present invention so as not to obscure the disclosure with details that will be readily apparent to those of ordinary skill in the art having benefit of the description herein.

Accordingly, to accomplish the objectives of the invention, the inventors disclose a high-throughput process for efficient production furano-naphthoquinone and pyrazinone derivatives using a novel strain Amycolatopsis sp. WGS_07. The broth may contain compounds other than the ones isolated and characterized herein and the disclosure should not be construed to be limited to the four evaluated compounds only.

The isolated strain of Amycolatopsis sp. WGS_07 was deposited at National Centre for Microbial Resource, Pune, India under accession No. MCC 0218.

In an embodiment of the present invention, Amycolatopsis sp. WGS_07 shows 99.24% similarity to that of strain Amycolatopsis silviterrae and 99.1% to strain Amycolatopsis vancoresmycina.

In another embodiment of the present invention, the crude extract of Amycolatopsis sp. WGS_07 is found to be active in 5254 and 5294 fermentation medium only with static and agar plate conditions against S. aureus.

In still another embodiment of the present invention, the naphthoquinone and pyrazinone based compounds synthesized from the Amycolatopsis sp. WGS_07 is having activity against gram-positive bacterial infections, malarial infections and cancer.

In yet another embodiment of the present invention, the naphthoquinone and pyrazinone based compounds are selected from, but not limited to:

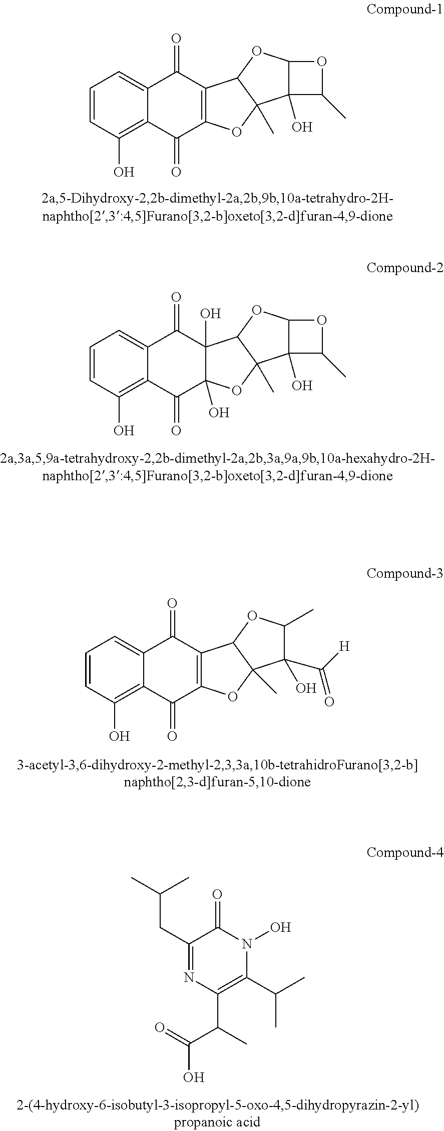

In another embodiment, the present invention relates to a process for the synthesis of naphthoquinone compounds from the isolated strain of Amycolatopsis sp. WGS_07, wherein the process comprises the steps of:
(a) isolating the strain Amycolatopsis sp. WGS_07 on MGYP agar plates for a period of 5-7 days at a temperature in a range of 25-30° C. to obtain the Amycolatopsis sp. WGS_07;
(b) fermenting the Amycolatopsis sp. WGS_07 using fermentation media in static condition for a period of 5-7 days at a temperature in a range of 25-30° C. to obtain broth mixture at a pH in a range of 6.8 to 7.2;
(c) extracting the broth mixture using suitable solvent to obtain an organic phase and concentrating the organic phase to obtain a crude extract comprising naphthoquinone derivatives;
(d) purifying the crude extract using column chromatography to obtain fractions comprising naphthoquinone derivative; and
(e) subjecting the fractions into the preparative HPLC to obtain the pure naphthoquinone compounds selected from:

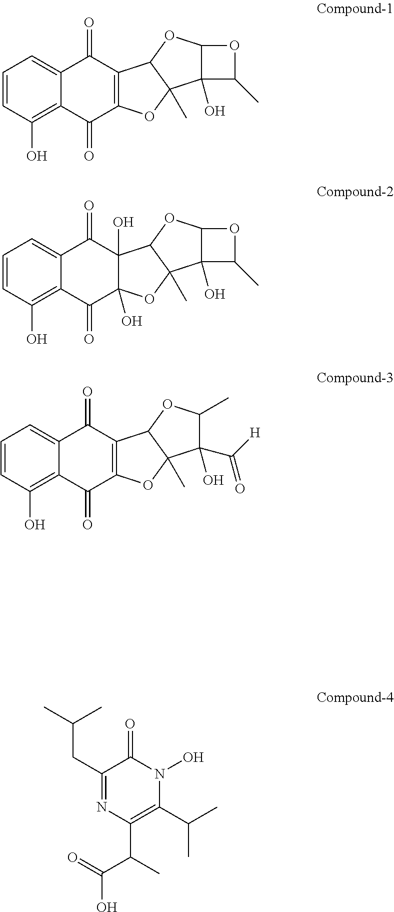

In another embodiment of the present invention, the isolation is carried out in a medium comprising 0.3% malt extract, 1% dextrose, 0.3% yeast, 0.5% peptone and 1.8% agar, pH 7.0.

In another embodiment of the present invention, the fermentation media is composed of a) 2% soy meal, 2% glucose, 0.5% corn steep liquor, 0.1% NaCl and 0.02% CaCO₃; or b) 1% starch, 1% glycerol, 1% dextrose, 0.2% yeast extract, 0.2% peptone, 0.25% corn steep liquor, 0.1% NaCl, and 0.3% CaCO₃.

In another embodiment of the present invention, the solvent is ethyl acetate, n-butanol, chloroform and dichloromethane.

In an embodiment of the present invention, the furano-naphthoquinone compound C-1 shows R, R, R, S, and R configurations at C1, C2, C3, C4, and C5 positions, respectively.

In another embodiment of the present invention, the pyrazinone derivatives C-4 shows S configurations at C8A, C8B, C8C, and C8D positions in four different conformers of C-4.

In an embodiment, the naphthoquinone compounds, C-1 to C-4 exhibit antibacterial activity.

In an embodiment of the present invention, the compounds C-1 and C-3 exhibit potent antibacterial activity.

In another embodiment of the present invention, the compounds C-1 and C-3 exhibit activity against resistant clinical bacterial strains.

In still another embodiment of the present invention, the compounds C-2 and C-4 are weakly active against gram-positive bacteria.

In yet another embodiment of the present invention, the naphthoquinone compounds do not exhibit any activity against gram-negative bacteria in the highest test concentration i.e. 128 μg/ml.

In an embodiment of the present invention, the compound C-4 exhibits antimalarial activity.

In another embodiment of the present invention, the compounds C-1 and C-3 exhibit potent antimalarial activity below 1 μM.

In still another embodiment of the present invention, the compounds C-2 and C-4 are moderately active against Plasmodium falciparum strain.

In another embodiment of the present invention, the compound C-1 and C-3 exhibits potent anticancer activity against A549 (lung cancer), HeLa (Cervical cancer) and MDA MB-231 (Breast cancer) cell lines.

In still another embodiment of the present invention, the compound C-2 and C-4 exhibits moderate anticancer activity against A549, HeLa and MDA MB-231 cell lines.

In yet another embodiment, the present invention relates to a pharmaceutical composition comprising naphthoquinone compounds useful for the treatment of gram-positive bacterial infections, malarial infections or cancer.

In yet another embodiment, the present invention relates to a pharmaceutical composition comprising the compounds or a stereoisomer, a tautomer, a pharmaceutically acceptable salt or a pharmaceutically acceptable solvate thereof as disclosed herein and one or more pharmaceutically acceptable excipients. In one another embodiment, the pharmaceutical composition is useful for the treatment of gram-positive bacterial infections, malarial infections, or cancer. In more embodiment, the pharmaceutical composition is in the form of a tablet, a capsule, solution, a gel, a suspension or a powder.

In another embodiment, the pharmaceutical compositions can be administered orally, for example in the form of pills, tablets, coated tablets, capsules, granules or elixirs. Administration, however, can also be carried out rectally, for example in the form of suppositories, or parenterally, for example intravenously, intramuscularly or subcutaneously, in the form of injectable sterile solutions or suspensions, or topically, for example in the form of ointments or creams or transdermally, in the form of patches, or in other ways, for example in the form of aerosols or nasal sprays.

In another embodiment, the present invention relates to a method of treating bacterial diseases comprising administering therapeutically effective amount of naphthoquinone compounds to a patient in need thereof.

In another embodiment, the present invention relates to a method of treating cancer comprising administering therapeutically effective amount of naphthoquinone compounds to a patient in need thereof.

In another embodiment, the present invention relates to naphthoquinone compounds for use in the treatment of bacterial diseases.

In another embodiment, the present invention relates to naphthoquinone compounds for use in the treatment of cancer.

Although the subject matter has been described in considerable detail with reference to certain examples and implementations thereof, other implementations are possible.

### EXAMPLES

The following examples, which include preferred embodiments, will serve to illustrate the practice of this invention, it being understood that the particulars shown are by way of example and for the purpose of illustrative discussion of preferred embodiments of the invention.

### Example 1:—Isolation of Amycolatopsis sp. WGS_07

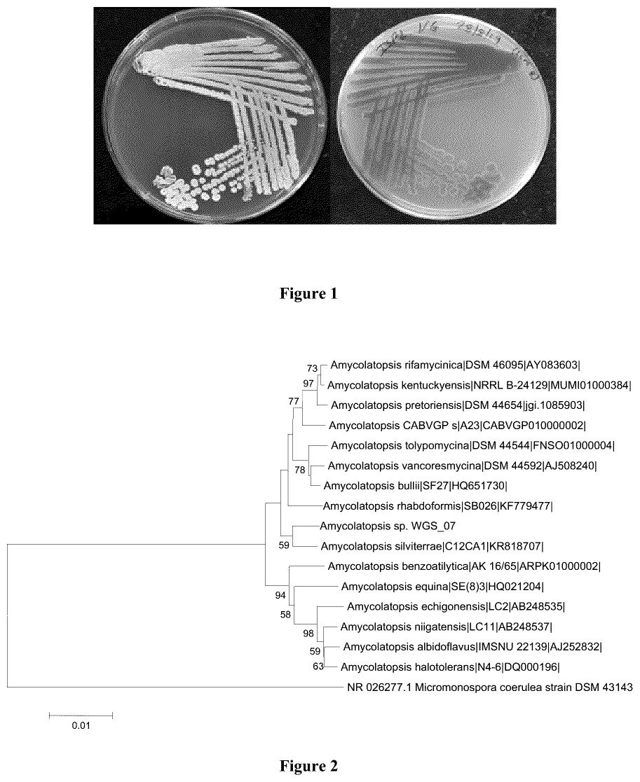

Amycolatopsis sp. WGS_07 was isolated from soil collected from the Western Ghats of Kerala, India. For the isolation of bacteria, a serial dilution method was followed and 100 μl of dilutions were spread on ISP-2 (1% malt extract, 0.4% yeast extract, 0.4% dextrose, pH 7.0) agar plates and incubated for 10 days at 28° C. The isolated culture was further sub-cultured on ISP-2 agar plates and 25% glycerol stocks were made for short-term storage and lyophilized vials were made for long-term storage (FIG. 1).

The isolated strain of Amycolatopsis sp. WGS_07 was deposited at National Centre for Microbial Resource, Pune, India under accession No. MCC 0218.

### Example 2:—Characterization of Amycolatopsis sp. WGS_07

The molecular identification of the strain was done by a 16S rRNA gene sequence and identified as Amycolatopsis sp. (GenBank No. MZ824481) showing 99.24% similarity to that of strain Amycolatopsis silviterrae (GenBank No.KR818707) and 99.1% to Amycolatopsis vancoresmycina (GenBank No. NR_025565). The top closest 15 cultures from the EZ-Biocloud database were used for the phylogenetic tree construction by the Neighbour Joining method using Mega 6.0 software with 1000 bootstrap values. The Amycolatopsis sp. WGS_07 was forming a clade with Amycolatopsis silviterrae (FIG. 2).

### Process of Synthesis of Compounds

### Example 3:—Bioactivity Screening of Amycolatopsis sp. WGS_07

Strain Amycolatopsis Sp. WGS_07 was sub-cultured on MGYP agar plates (0.3% malt extract, 1% dextrose, 0.3% yeast, 0.5% peptone and 1.8% agar, pH 7.0) for 7 days at 28° C. Loopful of culture was inoculated in a 250 ml Erlenmeyer flask containing 50 ml of seed medium, composed of 2% soy meal, 2% mannitol, 0.4% dextrose, and pH was adjusted to 7 before sterilization. The seed culture was incubated at 28° C. on a rotary shaker at 150 rpm for 3 days. 5 ml of the seed culture was used to inoculate the 3 different fermentation media in a 250 ml Erlenmeyer flask each containing 50 ml of media. The seed culture was also streaked on the fermentation medium agar plates containing 30 ml of media with 2% agar.

The fermentation media used were 5333 (composed of 1.5% starch, 0.4% yeast extract, 0.1% K₂HPO₄ and 0.05% MgSO₄), 5254 (composed of 2% soy meal, 2% glucose, 0.5% corn steep liquor, 0.1% NaCl and 0.02% CaCO₃) and 5294 (composed of 1% starch, 1% glycerol, 1% dextrose, 0.2% yeast extract, 0.2% peptone, 0.25% corn steep liquor, 0.1% NaCl, 0.3% CaCO₃). The pH of all three fermentation medium was adjusted to 7.0 prior to sterilization (autoclave 121° C. for 20 minutes). The flasks were incubated both in static condition and shaking condition (on a rotary shaker at 150 rpm) for 7 days at 28° C. The whole broth and agar plate with cultures were extracted by 100 ml of ethyl acetate and the organic phase was separated and concentrated by a rotary evaporator. The crude extract was dissolved in 1 ml of HPLC-grade ethyl acetate. For the bioactivity screening, 30 μl of crude extract was used against S.aureus, E.coli, and C. albicans by disc diffusion method.

Day wise activity of the supernatant of culture was performed by using 100 μl of cell free supernatant using the fermentation media 5294 in a 500 ml flask containing 100 ml of media.

### Results

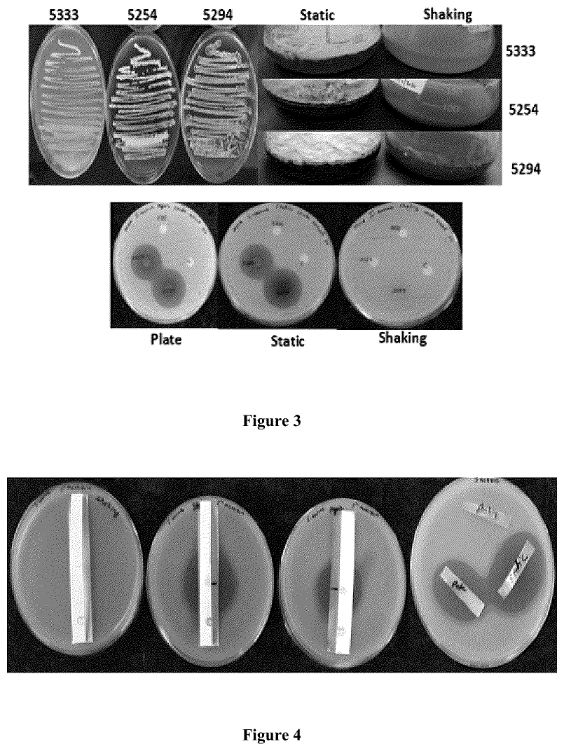

The crude extract of Amycolatopsis sp. WGS_07 was found to be active in 5254 and 5294 fermentation mediums only in static and agar plate conditions against S.aureus. No activity was observed in shaking conditions, refer Table 1 and FIG. 3.

TABLE 1
Activity of crude extract against S.aureus
(zone of inhibition in mm)

| Medium | Plate | Static | Shaking |
|---|---|---|---|
| 5333 | — | — | — |
| 5254 | 20 | 22 | — |
| 5294 | 22 | 25 | — |

Bioautography of the crude extract was done against S.aureus to check the major active compound zone in both the static and agar condition; hereby found that the major active compound in both the conditions is the same. Both the yellow pigment and zone of inhibition in shaking condition incubation were absent (FIG. 4).

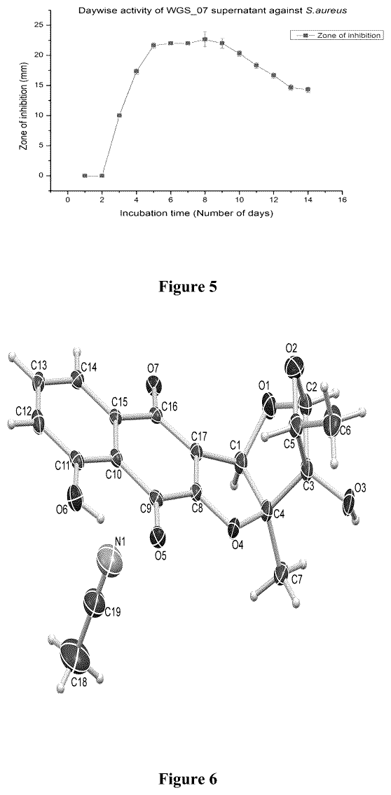

Day wise activity of Amycolatopsis sp.WGS_07 supernatant against S.aureus can be derived from FIG. 5.

### Example 4:—Fermentation, Extraction and Isolation of Compounds

The large-scale fermentation of Amycolatopsis sp. WGS_07 was carried out in a 500 ml Erlenmeyer flask containing 100 ml of 5294 fermentation medium and incubated in static condition for 7 days at 28° C. The whole broth was extracted with an equal volume of ethyl acetate 3 times. The organic phase was separated and concentrated under reduced pressure to get a semi-solid crude extract of about 52 grams. The extract was subject to silica gel column chromatography eluting with pet ether and ethyl acetate solvent system to give 20 fractions. Based on the bioactivity profile, fraction-9 which was eluted at 70% ethyl acetate and pet ether was further purified by semi-preparative HPLC on a C18 column using gradient elution by water and acetonitrile. It yielded 6 fractions of which Fraction 9.4 was found to be pure and was a major compound-1. Fraction-9.3 was further purified by semi-preparative HPLC to yield compound-2.

Based on bioactivity column fraction 5-8 was combined and silica gel column chromatography was done again by pet ether and ethyl acetate in gradient elution and the fraction_5-8_F-6 eluted at 30% ethyl acetate: pet ether was further separated by semi-preparative TLC in 20% ethyl acetate: pet ether followed by semi preparative HPLC in chiral amylose column in normal phase with isocratic mobile phase 30% isopropanol: hexane to yield compound-3. Compound-4 was purified by semi-preparative HPLC by reverse phase C18 column using a gradient mobile phase acetonitrile and water from fraction_5-8_F-5 eluted at 20% ethyl acetate: pet ether in silica gel column chromatography. Table 2 exhibits the yield of the purified compounds from 50 litres of fermentation.

TABLE 2
Yield of the purified compounds from 50 litres of fermentation

| Compounds | Yield (mg) in 50 litres |
|---|---|
| Compound 1 | 5250 mg |
| Compound 2 | 186 mg |
| Compound 3 | 32 mg |
| Compound 4 | 146 mg |

### Example 5:—Characterization of Isolated Compounds

### Compounds 1, 2, 3 and 4 Structure, IUPAC Name, Process of Synthesis and Characterization

A major active purified compound was in an isomeric form containing chiral carbons so to get its exact structure and absolute configurations, recrystallization of the compound C-1 was performed to get a pure crystal. Molecular formula and molecular mass of the derived compounds can be found in Table 3.

TABLE 3
Molecular formula and molecular mass of the compounds

| Sample | Molecular formula | m/z (M + H)⁺ | Mass |
|---|---|---|---|
| C-1 | C₁₇H₁₄O₇ | 331.0802 | 330.0732 |
| C-2 | C₁₇H₁₆O₉ | 365.0858 | 364.0787 |
| C-3 | C₁₇H₁₄O₇ | 331.0803 | 330.0733 |
| C-4 | C₁₄H₂₂N₂O₄ | 283.165 | 282.1578 |

### Optical Rotation

Optical rotation was done on Jasco P-2000 polarimeter instrument, and the samples were dissolved in Acetonitrile, the specific optical rotation of the derived compounds can be referred from Table 4.

TABLE 4
Specific optical rotation of the compounds

| | | | Specific Optical |
|---|---|---|---|
| Sample | Temperature | Concentration | Rotation |
| C-1 | 27.12 | 0.6 | 97.846 |
| C-2 | 26.86 | 0.53 | 93.358 |
| C-3 | 26.94 | 0.26 | 84.261 |
| C-4 | 27.03 | 0.54 | 31.311 |

### NMR Based Characterization of Compounds

The purified compounds were applied to 1D and 2D NMR analysis to elucidate the exact structure of naphthoquinone derivatives and are listed in Table 5.

TABLE 5
NMR table (¹H and ¹³C) of purified compounds

| | C-1 | | C-2 | | C-3 | | C-4 | |
|---|---|---|---|---|---|---|---|---|
| Position | δ_{H} (J in Hz) | δ_{C} | δ_{H} (J in Hz) | | δ_{H} (J in Hz) | δ_{C} | δ_{H} (J in Hz) | δ_{C} |
| 1 | 5.74 (s, 1 H) | 92.2 | 4.42 (s, 1 H), | 88.2 | 5.54 (s, 1 H) | 84.5 | | 151.1 |
| | | (CH) | | (CH) | | (CH) | | |
| 2 | 5.72 (s, 1 H) | 111.7 | 5.14 (s, 1 H), | 97.5 | 3.89 (q, J = | 77.7 | | 138.2 |
| | | (CH) | | (CH) | 6.4 Hz, 1H) | (CH) | | |
| 3 | | 100.2 | | 81.8 | | 88.6 | | 132 |
| 4 | | 87.4 | | 83.6 | | 98.7 | | 151.3 |
| 5 | 4.50 (q, J = | 75.9 | 4.09 (q, J = | 71.2 | 1.13 (d, J = | 13.3 | 3.42-3.28 | 26.7 |
| | 6.4 Hz, 1 H) | (CH) | 6.7 Hz, 1 H), | (CH) | 6.9 Hz, 3H) | (CH₃) | (m, 1H) | (CH) |
| 6 | 1.34 (d, J = | 17.9 | 1.07 (d, J = | 14.5 | 1.40 (s, 3 H) | 17.7 | 1.46 (d, J = | 19.1 |
| | 6.1 Hz, 3 H) | (CH₃) | 6.7 Hz, 3 H) | (CH₃) | | (CH₃) | 7.2 Hz, 3 H)^{a} | (CH₃) |
| 7 | 1.59 (s, 3 H) | 19.7 | 1.65 (s, 3 H) | 18.9 | 9.74 (s, 1 H) | 202.3 | 1.44 (s, J = | 18.9 |
| | | (CH₃) | | (CH₃) | | | 7.2 Hz, 3 H)^{a} | (CH₃) |
| 8 | | 161 | | 92.3 | | 162.3 | 3.98 (q, J = | 42.4 |
| | | | | | | | 6.8 Hz, 1 H) | (CH) |
| 9 | | 184 | | 194 | | 184 | 1.51(d, J = | 22.6 |
| | | | | | | | 7.2 Hz, 3 H) | (CH₃) |
| 10 | | 116.3 | | 117.4 | | 116.5 | | 177.9 |
| 11 | | 162.9 | | 163.3 | | 162.9 | 2.69 (d, J = | 41.5 |
| | | | | | | | 6.8 Hz, 2 H) | (CH₂) |
| 12 | 7.25 (d, J = | 124.9 | 7.29 (d, J = | 124.6 | 7.27 (dd, J = | 125 | 2.17 (m, 1 | 28.8 |
| | 8.4 Hz1 H) | (CH) | 8.2 Hz, | (CH) | 1.1, 8.4 Hz, 1 | (CH) | H) | (CH) |
| | | | 1H), | | H) | | | |
| 13 | 7.73-7.65 (m, | 138.6 | 7.74 (t, J = | 138.5 | 7.72 (dd, J = | 138.7 | 0.93 (s, J = | 17.6 |
| | 1 H) | (CH) | 7.6, Hz, | (CH) | 7.6, 8.4 Hz, | (CH) | 6.8 Hz, 3H)^{b} | (CH₃) |
| | | | 1H) | | 1H) | | | |
| 14 | 7.64-7.56 (m, | 119.9 | 7.64 (d, J = | 120.8 | 7.61 (dd, J = | 119.9 | 0.91 (s, J = | 17.6 |
| | 1 H) | (CH) | 7.6 Hz, 1 | (CH) | 1.0, 7.4 Hz, | (CH) | 6.8 Hz, 3H)^{b} | (CH₃) |
| | | | H), | | 1H), | | | |
| 15 | | 134.4 | | 137.2 | | 134.5 | | |
| 16 | | 181.6 | | 189.2 | | 181.8 | | |
| 17 | | 126.0 | | 104 | | 123.3 | | |
| 18- | 11.51 (br. | | 11.10 (br. | | 11.51 (br. | | 10.38 (br. | |
| | s, 1 H) | | s., 1 H) | | s., 1 H) | | s., 1H) | |
| 19 | | | | | | | 10.48 (br. | |
| | | | | | | | s., 1H) | |

### X-Ray Diffraction

X-ray intensity data measurements of the compound were carried out on a Bruker D8 VENTURE Kappa Duo PHOTON II CPAD diffractometer equipped with Incoatec multilayer mirrors optics. The intensity measurements were carried out with a Cu micro-focus sealed tube diffraction source (CuKα=1.54178 Å) at 100(2) K temperature.

### Results

C-1: The absolute configuration was established by anomalous dispersion effect (Flack parameter, 0.06(18)) in X-ray diffraction measurements carried out with Cu radiation. The single-crystal X-ray diffraction data analysis established that our synthesized compound has R, R, R, S, and R configurations at C1, C2, C3, C4, and C5 positions, respectively (FIG. 6). The displacement ellipsoids were drawn at the 50% probability level and H atoms were shown as small spheres with arbitrary radii.

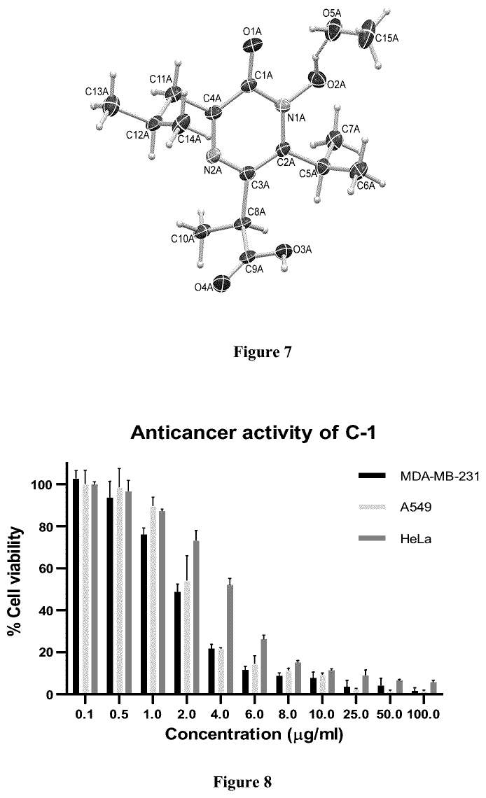

C-4: The single-crystal X-ray diffraction data analysis established that our synthesized compound has S configurations at C8A, C8B, C8C, and C8D positions in four different conformers of C-4 (FIG. 6). FIG. 7 depicts the ORTEP view of compound C-4 showing the atom-numbering scheme. The displacement ellipsoids are drawn at the 30% probability level and H atoms are shown as small spheres with arbitrary radii. The other molecules of C-4 present in the asymmetric unit are omitted for clarity.

### Example 5:—Antibacterial Activity of the Compounds

The MICs of the purified compounds were done according to CLSI guidelines using Mueller Hinton broth (Hi media) against panel of six Gram positive bacteria S.aureus, S.epidermidis, B.cereus, B.subtilis, M.luteus and L.monocytogenes, two Gram negative bacteria E.coli and P.aeruginosa, and four MRSA (Methicillin resistant S.aureus) clinical strain. The stock solution of compounds were made at 10 mg/ml in DMSO. The test compounds (highest concentration 128 μg/ml) were serially diluted in 50 μl of Mueller Hinton broth and 50 μl of bacterial suspension were added to reach the final desired cell density of 5×10⁵ CFU ml⁻¹ in each well of 96 well microtiter plate except the media control. The medium, untreated culture, DMSO, ampicillin and kanamycin were used as controls. The plates were observed for MIC after incubation of 18 hours at 37° C. in shaking condition. The MIC for Mycobacterium smegmatis was done by the same method using Middlebrook 7H9 medium (Hi media) and incubated for 48 hours at 37° C. in shaking condition. The MICs were defined as the lowest concentration that inhibited visible growth of bacteria.

Results: The compounds C-1 and C-3 were having good antibacterial activity whereas compounds C-2 and C-4 were weakly active against Gram-positive bacteria. The compounds C-1 and C-3 were also active against the resistant clinical bacterial strains. The compounds C-1 and C-3 were also found to be weakly active against Mycobacterium smegmatis. No activity was observed against Gram-negative bacteria by any of the 4 compounds in the highest test concentration (128 μg/ml) refer table 6.

TABLE 6
MIC of the purified compounds

| | MIC (μg/ml) | | | | | |
|---|---|---|---|---|---|---|
| Bacteria | C-1 | C- | C-3 | C-4 | Ampicilli | Kanamycin |
| Staphylococcus aureus ATCC 9144 | 2 | 12 | 8 | 128 | 0.03 | 8 |
| Staphylococcus epidermidis ATCC | 2 | 64 | 8 | 64 | 1 | 1 |
| Bacillus cereus ATCC 11778 | 8 | — | 16 | 128 | 16 | 2 |
| Bacillus subtilis ATCC 6633 | 4 | — | 16 | 128 | 0.03 | 0.5 |
| Micrococcus luteus ATCC 9341 | 8 | — | 32 | 64 | 0.03 | 8 |
| Listeria monocytogenes ATCC 19111 | 2 | 12 | 8 | 128 | 0.03 | 1 |
| Mycobacterium smegmatis ATCC 607 | 64 | — | 64 | — | 64 | 0.25 |
| MRSA_3B | 4 | — | 16 | 128 | 8 | — |
| MRSA_6B | 4 | — | 8 | 128 | 8 | — |
| MRSA_9B | 4 | — | 16 | 128 | 16 | 128 |
| MRSA_10B | 4 | 12 | 8 | 128 | 4 | — |
| Escherichia coli ATCC 8739 | — | — | — | — | 2 | 8 |
| Pseudomonas aeruginosa ATCC 9027 | — | — | — | — | 2 | 8 |

### Example 6:—Antimalarial Activity of the Compounds

The antimalarial activity was carried out on blood-stage Plasmodium falciparum (3D7 strain) with various concentrations ranging from 10 μM to 0.01 μM by 5 SYBR green staining-based parasitemia determination.

Parasite cultures were incubated with the test compounds for 60 h under optimal growth conditions, following which the cells were lysed with 0.01% Triton X and stained with SYBR Green I nucleic acid stain (Thermo Fisher Scientific) to estimate parasite growth and inhibition. Fluorescence readings were obtained with a GloMax plate reader (Promega) after 15 min incubation in the dark and raw fluorescence readings were processed. The % growth inhibition values were estimated from comparisons between test and control samples.

### Results

The compounds C-1 and C-3 were having potent antimalarial activity below 1 μM whereas the compounds C-2 and C-4 were moderately active against the tested Plasmodium falciparum strain refer table 7.

TABLE 7
Antimalarial activity of purified compounds

| | Antimalarial activity | |
|---|---|---|
| Compounds | EC50 ( [symbol] M) | Std. dev. |
| C-1 | 0 [symbol] 20 | 0 [symbol] 02 |
| C-2 | 4 [symbol] 60 | 0 [symbol] 51 |
| C-3 | 0 [symbol] 48 | 0 [symbol] 08 |
| C-4 | 7 [symbol] 79 | 0 [symbol] 19 |

[symbol]

indicates data missing or illegible when filed

### Example 7:—Anticancer Activity of the Compounds

MTT assay was used for the study of cell viability, which measured cellular metabolic activity based on the ability of nicotinamide adenine dinucleotide phosphate (NADPH) dependent oxidoreductase enzymes to reduce the yellow tetrazolium salt 3-[4,5-dimethylthiazole-2-yl]-2,5-diphenyltetrazolium bromide (MTT) to purple formazan crystals. The cell lines used for the study were A549 (lung cancer), HeLa (Cervical cancer) and MDA MB-231 (Breast cancer), which were seeded with a density of 10,000 cells/well into 96-well plates and incubated at 37° C. with 5% CO2 for 20 hours. The compounds with different concentrations were used to treat the cells for 24 hours. The medium was replaced with 100 μL of MTT solution (0.5 mg/mL in DMEM) and then incubated for 4 h at 37° C. in dark. Subsequently, MTT solution was removed and the formazan crystals were solubilized by addition of 100 μl DMSO. The optical density was measured at 570 nm by a Biotek synergy H1 microplate reader.

### Results

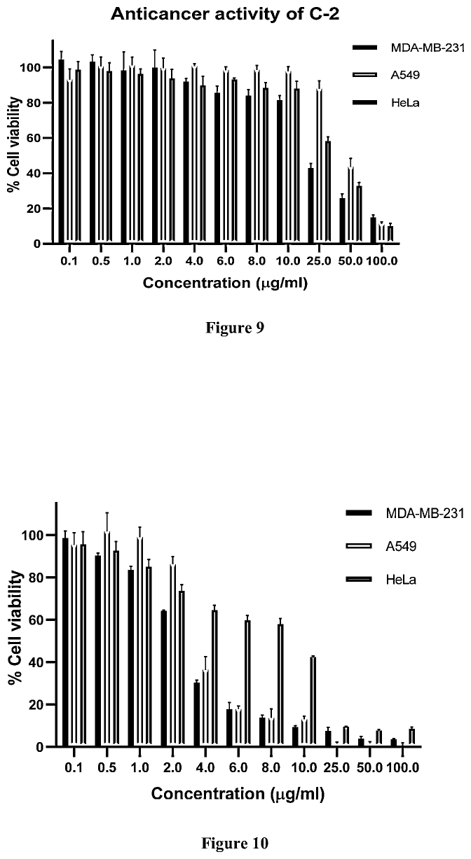

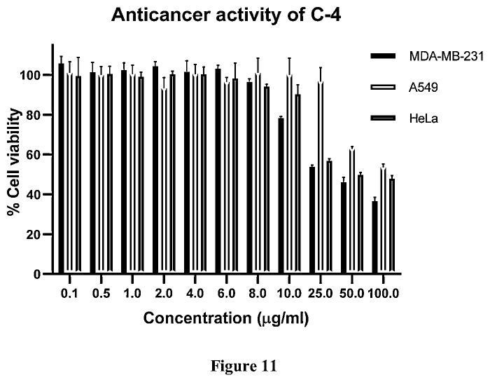

The compound C-1 and C-3 were showing potent inhibitory activity against the tested cancer cell lines MDA-MB-231, A549 and HeLa with IC₅₀ value in the range of 1.79-8.823 μg/ml whereas the compounds C-2 and C-4 were showing moderate activity refer table 8 and FIGS. 8 to 11.

TABLE 8
Anticancer activity of the purified compounds

| | IC₅₀ Value (μg/ml) | | |
|---|---|---|---|
| Compound | MDA-MB-231 | A549 | HeLa |
| C-1 | 1.790 | 2.141 | 3.534 |
| C-2 | 19.09 | 44.86 | 35.97 |
| C-3 | 2.598 | 3.380 | 8.823 |
| C-4 | 14.11 | 40.21 | 15.40 |

### ADVANTAGES OF THE PRESENT INVENTION

The present invention provides a process for high yield production of naphthoquinone derivatives from a novel bacterial strain.
The process disclosed herein for preparing naphthoquinone derivatives is cost-effective.
The process disclosed herein for the production of naphthoquinone derivatives exhibits activities against gram-positive bacterial infections, malarial infections, and cancer.

We claim:

1. An isolated strain of Amycolatopsis sp. WGS_07 having accession number MCC 0218.

2. The isolated strain as claimed in claim 1, wherein the Amycolatopsis sp. WGS_07 shows 99.24% similarity to that of strain Amycolatopsis silviterrae and 99.1% to strain Amycolatopsis vancoresmycina.

3. The isolated strain as claimed in claim 1, wherein the Amycolatopsis sp. WGS_07 is isolated from soil using serial dilution method in which 100 μl of dilutions is spread on ISP-2 (1% malt extract, 0.4% yeast extract, 0.4% dextrose, pH 7.0) agar plates and incubated for 10 days at 28° C.

4. A process for the synthesis of naphthoquinone and pyrazinone based compounds from the isolated strain of Amycolatopsis sp. WGS_07 as claimed in claim 1, wherein the process comprises the steps of:
(a) isolating the strain Amycolatopsis sp. WGS_07 on MGYP agar plates for a period of 5-7 days at a temperature in a range of 25-30° C. to obtain the Amycolatopsis sp. WGS_07;
(b) fermenting the Amycolatopsis sp. WGS_07 using fermentation media in static condition for a period of 5-7 days at a temperature in a range of 25-30° C. to obtain broth mixture at a pH in a range of 6.8 to 7.2;
(c) extracting the broth mixture using suitable solvent to obtain an organic phase and concentrating the organic phase to obtain a crude extract comprising naphthoquinone derivatives;
(d) purifying the crude extract using column chromatography to obtain fractions comprising naphthoquinone derivative; and
(e) subjecting the fractions into the preparative HPLC to obtain the pure naphthoquinone and pyrazinone based compounds selected from:

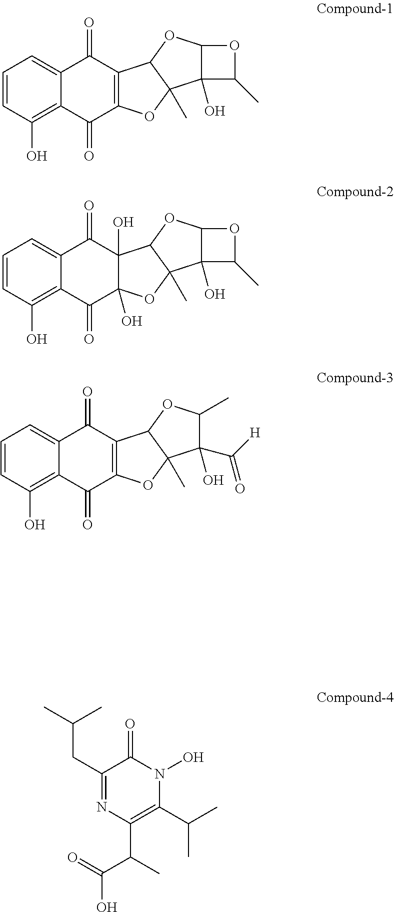

5. The process as claimed in claim 4, wherein the isolating is carried out in a medium comprising 0.3% malt extract, 1% dextrose, 0.3% yeast, 0.5% peptone and 1.8% agar, at a pH of 7.0.

6. The process as claimed in claim 4, wherein the fermentation media is composed of 2% soy meal, 2% glucose, 0.5% corn steep liquor, 0.1% NaCl and 0.02% CaCO₃; or 1% starch, 1% glycerol, 1% dextrose, 0.2% yeast extract, 0.2% peptone, 0.25% corn steep liquor, 0.1% NaCl, and 0.3% CaCO₃.

7. The process as claimed in claim 4, wherein the solvent is ethyl acetate, n-butanol, chloroform and dichloromethane.

8. A naphthoquinone and pyrazinone based compound is selected from the group consisting of:

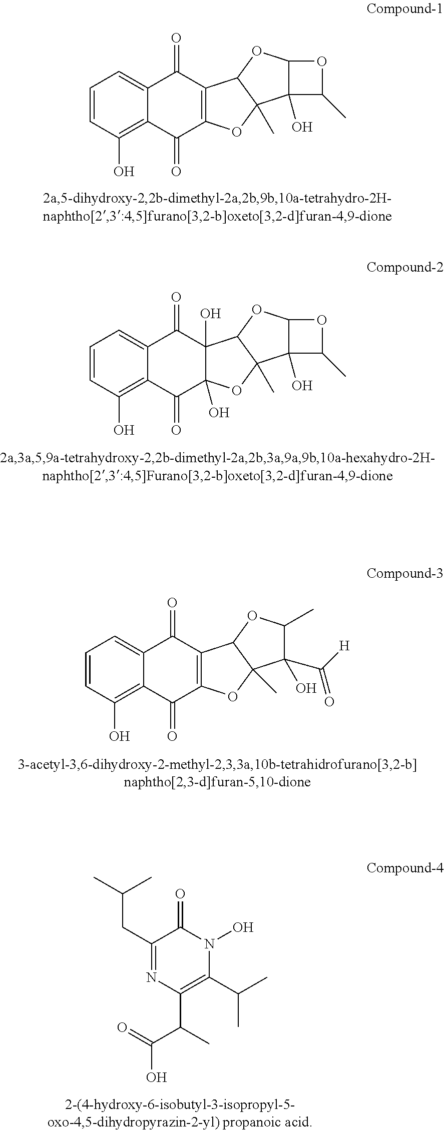

9. A pharmaceutical composition comprising the compounds or a stereoisomer, a tautomer, a pharmaceutically acceptable salt or a pharmaceutically acceptable solvate thereof as claimed in claim 8 and one or more pharmaceutically acceptable excipients.

10. The pharmaceutical composition as claimed in claim 9, wherein the pharmaceutical composition is useful for the treatment of gram-positive bacterial infections, malarial infections or cancer.

11. The pharmaceutical composition as claimed in claim 9, wherein the pharmaceutical composition is in the form of a tablet, a capsule, solution, a gel, a suspension or a powder.
